(19) United States
(12) Patent Application Publication
(10) Pub. No.: US 2022/0096595 A1
(43) Pub. Date: Mar. 31, 2022

(54) ANTI-ATOPIC DERMATITIS PROTEIN

(71) Applicant: ZHENGZHOU UNIVERSITY, Zhengzhou (CN)

(72) Inventors: Qiaozhen KANG, Zhengzhou (CN); Xin LIU, Zhengzhou (CN); Jike LU, Zhengzhou (CN); Zhenyu JI, Zhengzhou (CN); Ting WANG, Zhengzhou (CN); Juanjuan YI, Zhengzhou (CN); Chenglong ZHANG, Zhengzhou (CN); Xun GUO, Zhengzhou (CN)

(73) Assignee: ZHENGZHOU UNIVERSITY, Zhengzhou (CN)

(21) Appl. No.: 17/427,109

(22) PCT Filed: Mar. 30, 2020
(86) PCT No.: PCT/CN2020/082001

§ 371 Date: Jul. 30, 2021

(30) Foreign Application Priority Data
Jan. 30, 2019 (CN) 201910089815.4
Jan. 30, 2019 (CN) 201910089839.X

(51) Int. Cl. A61K 38/17; A61P 17/04

(52) U.S. Cl. CPC A61K 38/17; A61P 17/04

(57) ABSTRACT

Provided is an anti-atopic dermatitis protein. A corresponding pharmaceutical composition contains a pharmaceutically acceptable carrier and the anti-atopic dermatitis protein. The anti-atopic dermatitis protein is one or more proteins selected from the group consisting of Helicobacter pylori-neutrophil-activating protein (HP-NAP) and recombinant maltose-binding protein fused to neutrophil-activating protein (rMBP-NAP). HP-NAP and rMBP-NAP can effectively treat AD in an oxazolone-induced AD model, providing brand-new drugs for the treatment of AD.

### CROSS REFERENCE TO THE RELATED APPLICATIONS

This application is the national phase entry of International Application No. PCT/CN2020/082001, filed on Mar. 30, 2020, which is based upon and claims priority to Chinese Patent Application No. 201910089839.X, filed on Jan. 30, 2019, and Chinese Patent Application No. 201910089815.4, filed on Jan. 30, 2019, the entire contents of which are incorporated herein by reference.

### TECHNICAL FIELD

This invention relates to biotechnology.

### BACKGROUND

Atopic Dermatitis (AD) is a chronic skin disease characterized by dryness, itching, erythema eczema and selective accumulation of inflammatory cells. The condition of AD is easy to repeat and difficult to cure, which seriously affects the patient's health and quality of life. The pathogenesis of AD is a result of the combined actions of genetic inheritance, environmental factors, skin barrier function defects, immune abnormalities, etc., and has not been fully elucidated so far. In recent years, the incidence of AD has increased year by year, and the treatment of AD has become an important issue that has attracted much attention. At present, hormone medicines such as antihistamine and steroid hormone are mostly used for treating AD, and side effects such as drug resistance and skin atrophy are generated after long-term use.

The virulence factors of Helicobacter pylori include Helicobacter pylori-neutrophil-activating protein (HP-NAP), CagA, CagPAI, VacA, OipA, BabA, etc., all of which can cause inflammatory reaction. Our laboratory has submitted the coding gene sequence of HP-NAP (Genebank accession number AY366361), and has cloned and expressed the gene sequence by using genetic engineering technology to obtain helicobacter pylori-neutrophil activating protein (HP-NAP). In addition, we has fused Maltose Binding Protein (MBP) with HP-NAP by using genetic engineering technology to obtain a fusion protein rMBP-NAP, and a report related to the fusion protein is as follows:

Wang, T., et al., International Immunopharmacology, 29.2(2015): 876-883.

### SUMMARY

The present invention discloses an application of HP-NAP (helicobacter pylori-neutrophil activating protein) in treating atopic dermatitis, the coding gene of which can be obtained by querying the accession number AY366361 in the Genebank; the invention also discloses an application of the fusion protein of HP-NAP and MBP, rMBP-NAP, in treating atopic dermatitis. In the present invention, the HP-NAP and rMBP-NAP can also be used in combination for treating atopic dermatitis.

The present invention also correspondingly discloses an anti-atopic dermatitis pharmaceutical composition comprising the above-mentioned active protein or proteins, which comprises a pharmaceutically acceptable carrier. The dosage form of the pharmaceutical composition can be injection, such as powder for injection or solution for injection, and the route of administration can be intraperitoneal injection.

The applicant discovers that HP-NAP and rMBP-NAP can effectively treat AD in an oxazolone-induced AD model, providing brand-new drugs for the treatment of AD.

### BRIEF DESCRIPTION OF THE DRAWINGS

FIG. 1A is an analysis of the ear thickness of all the groups in the HP-NAP anti-AD experiment;

FIG. 1B is an analysis of photographs of AD mice taken in whole body and ear for each experimental group of the HP-NAP anti-AD experiment, with arrows showing local enlargement of ear;

FIG. 2A is a statistical analysis of the thickness of the epidermal layer of ear tissue in each experimental group of the HP-NAP anti-AD experiment;

FIG. 2B is a statistical analysis of the number of ear mast cells in each experimental group of the HP-NAP anti-AD experiment;

FIG. 3 is an analysis of the ear thickness of all the groups in the rMBP-NAP anti-AD experiment;

FIG. 4A is a statistical analysis of the thickness of the ear epidermis of each experimental group of mice in the rMBP-NAP anti-AD experiment;

FIG. 4B is a statistical analysis of the number of ear mast cells in each experimental group of the rMBP-NAP anti-AD experiment;

FIG. 5 is a statistical analysis of mice liver weight after administration for each experimental group of the rMBP-NAP anti-AD experiment;

the figures above may refer to the unified meaning of the figures: * denotes P<0.05, ** denotes P<0.01, *** denotes P<0.001.

### DETAILED DESCRIPTION OF THE EMBODIMENTS

### I. Therapeutic Effect of the Protein HP-NAP on OXA-Induced AD Mice Model

The experimental method comprises the following steps:

AD mice model dosing:

a. BALB/c mice, female and aged 7 weeks, were purchased. After kept in the laboratory for one week, the mice were randomly divided into 4 groups—Control Group, Sensitization Group, HP-NAP Administration Group and Dexamethasone(DEX) Administration Group (6 in each group). The mice were kept in independent ventilated cages, and labeled.

b. The back of the 8-week-old BALB/c mice was shaved to an area of about 2 cm×2 cm, and after 24 hours, the BALB/c mice were sensitized by smearing 20 μL of sensitizing solution containing 5% Oxazolone onto their backs, while the mice in the Control Group were treated with a mixture (20 μL) of acetone and olive oil.

c. One week later, 20 μL of 0.3% oxazolone solution was smeared onto the medial side of the mice ear for ear challenge (the Control Group was treated with acetone:olive oil=4:1 alternatively) three times per week.

d. HP-NAP dosing regimen:

Intraperitoneal administration was started on day 0 after the OXA-induced AD model was established, 3 times a week for 7 times, 200 μg/0.2 mL per dose. On days 0-14, one hour after the “ear challenge” each time, the mice in each group were dosed separately as follows: the Sensitization Group was injected with 200 μL PBS solution intraperitoneally; the HP-NAP Administration Group was injected with a HP-NAP solution(200 μg/0.2 mL) intraperitoneally; the DEX Administration Group was injected with a dexamethasone (DEX) solution(200 μg/0.2 mL) intraperitoneally. The intraperitoneal administration was done three doses per week, and the mice were sacrificed on day 16.

Detecting the skin damage severity of AD mice:

Between day −7 to day 16, the thickness of auricle in mice is measured by a thickness gauge every day before the sensitization or the “ear challenge”. The skin damage condition of the mice in the each group of the AD model is observed and photographed.

Histopathological examination of AD mice:

a. On the 16th day of the onset of the AD model mice, the mice were sacrificed with anesthetic. The disease ear tissues of the BALB/c mice were cut off and fixed in the fixing solution of 4% paraformaldehyde for more than 24 hours.

b. The ear tissue of the mice was embedded in paraffin and cut into 6 μm sections, and then stained with H&E. After the H&E sections are photographed, the photographs are analyzed by using the software ImageJ, and the ear epidermis thickness is calculated.

c. The ear tissue of the mice was embedded in paraffin and cut into 6 μm sections and stained with toluidine blue. After the sections are photographed, the photographs are analyzed by ImageJ, and the infiltration of inflammatory cells in the ear tissues is determined.

The experimental results are as follows:

1. Amelioration of Symptoms of Atopic Dermatitis in Mice by HP-NAP

The Sensitization Group:

From the 7th day, the ear swelling of the mice increased significantly, and the ear redness-swelling degree became severe till the day 14-16, scabs gradually formed on the ear and lichen sclerosus occurred then.

The HP-NAP Administration Group:

Compared with the Sensitization Group, on the 14th-16th day, the ear of the AD mice had no scabbing and the red ness-swelling was significantly suppressed, and the ear thickness was reduced.

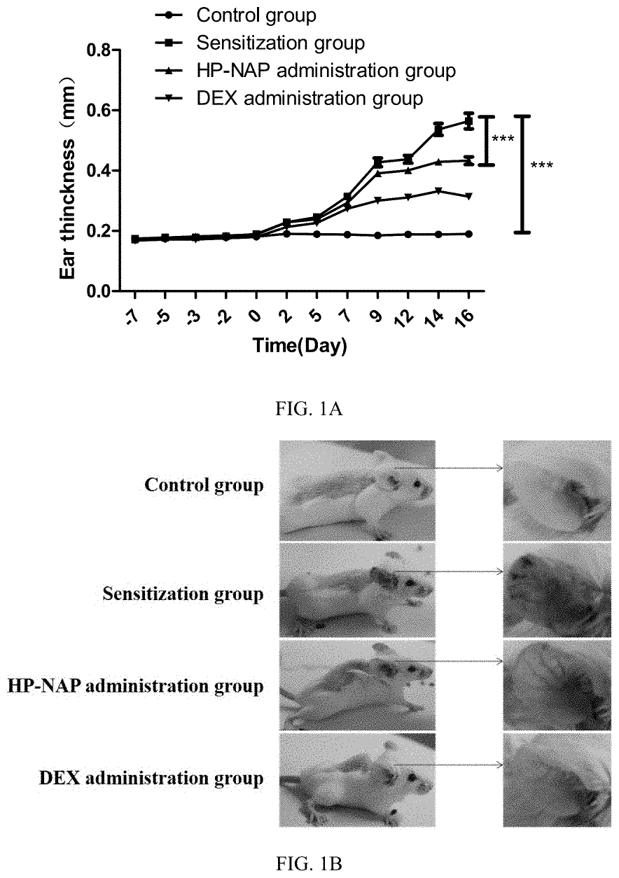

Herein, the data on the thickness of the ears are shown in FIG. 1A, and the ear photograph of the mice before sacrificed is shown in FIG. 1B. It is clear that the results of the HP-NAP Administration Group are significantly different from the Sensitization Group.

2. Histopathological Examination of AD Mice

H&E analysis shows:

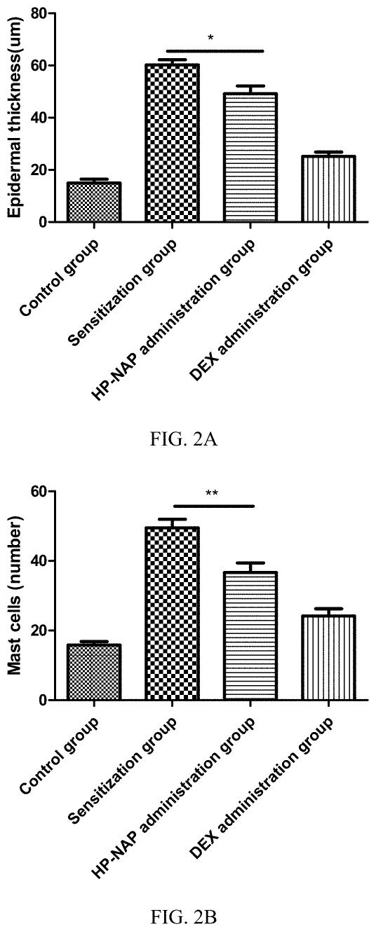

Symptoms occurred in the Sensitization Group comprising that stratum corneum of the skin was damaged, the dermis was thickened, a large number of inflammatory cells infiltrated, and the blood vessels were dilated. But in the HP-NAP Administration Group, the exudation of the inflammatory cells was not obvious, the epidermis was slightly thickened, the stratum corneum was intact, and the blood vessels were not obviously dilated, which suggest that the therapeutic effect of the HP-NAP Administration Group is more significant (see FIG. 2A).

The ear tissues were stained with toluidine blue to observe the infiltration of mast cells in the ear tissues of the Sensitization Group. The results showed that in the Sensitization Group, near the ear epidermis layer, there were a large number of purple or purplish red granules (those indicates the mast cells stained). But, toluidine blue staining analysis of the ear in the HP-NAP Administration Group showed that: the number of the mast cells near the epidermis of the ear was decreased and the infiltration of the mast cells was reduced (see FIG. 2B).

### II. Therapeutic Effect of the Protein rMBP-NAP on the Mice Model of OXA-Induced AD

The experimental method comprises the following steps:

AD mice model dosing:

a. The back of 7-week-old BALB/c mice were shaved to an area of about 2 cm² , and after 12 hours, the BALB/c mice were sensitized by smearing 20 μL of sensitizing solution containing 5% oxazolone onto their backs, while the mice in the Control Group were treated with a mixture(20 μL) of acetone and olive oil.

b. One week later, 20 μL of 0.3% of oxazolone solution was smeared onto the medial side of the mice ear for ear challenge (the Control Group was treated with acetone:olive oil=4:1 alternatively) three times per week.

c. Mice were randomly divided into 4 groups, including Control Group, Sensitization Group, rMBP-NAP Administration Group, DEX Administration Group (6 in each group), raised in independent ventilated cages, and labeled.

d. Dosing regimen for the fusion protein rMBP-NAP:

Intraperitoneal administration was started on day 0 after the mice model of OXA-induced AD was established, three times a week for ten times, 200 μg/0.2 mL per dose. On days 0-21, one hour after the “ear challenge” each time, the mice in each group were dosed individually as follows: the Sensitization Group is injected with 200 μL PBS solution intraperitoneally; the rMBP-NAP Administration Group is injected with a rMBP-NAP solution (200 μg/0.2 mL) intraperitoneally; the DEX Administration Group was injected with a dexamethasone (DEX) solution (200 μg/0.2 mL) intraperitoneally. The intraperitoneal administration was done three doses per week, and the mice were sacrificed on day 22 of the experiment.

Detecting the skin damage severity of AD mice:

Between day −7 to day 22, the thickness of auricle in mice is measured by a thickness gauge every day before the sensitization or the “ear challenge”. The skin damage of the mice in the each group of the AD model is observed and photographed.

Histopathological examination of the AD mice:

a. On the “peak incidence” (day 16) of the AD mice model, the mice were sacrificed with anesthetic. The diseased ear tissues of the BALB/c mice were cut off, and fixed in the fixing solution of 4% paraformaldehyde for more than 24 h.

b. The ear tissue of the mice was embedded in paraffin and cut into 6μm sections, and then all stained with H&E. Microscope was used for histopathological observation of the ear tissues.

c. After the ear H & E section is photographed, the photograph is analyzed by using ImageJ, and the infiltration condition of inflammatory cells in ear tissues is determined.

Organ index analysis of AD mice:

After the AD model mice were dosed, they were euthanized by injection of 200 μL of 1% sodium pentobarbital on day 22. The mice were dissected at their abdomen to get their spleen tissue and liver tissue. The spleen tissue and the liver tissue were weighed after the residual liquid on them was soaked up by absorbent paper, and the data was recorded. The organ index differences of the AD mice dosed in all the groups were statistically analyzed.

The experimental results are as follows:

1. Amelioration of Symptoms of Atopic Dermatitis Mice by rMBP-NAP

The Sensitization Group:

The mice had increased ear swelling, scabs gradually formed on the ear, and lichen sclerosus occured. On days 15-22, the ear thickness increased, the ear redness-swelling degree was severe, and markedly scabbing occurred.

The rMBP-NAP Administration Group:

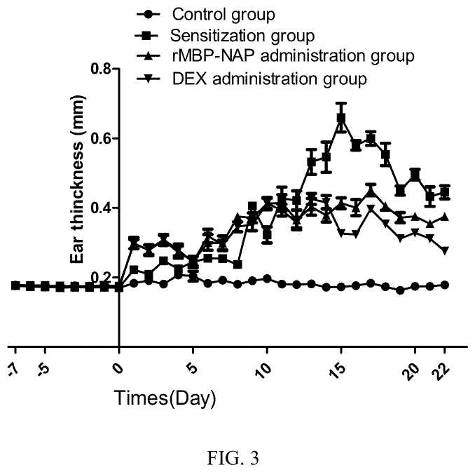

AD symptoms in the ears of the mice, the redness-swelling and scabbing were obviously suppressed, and the ear thickness was reduced. On the “peak incidence” (day 16), compared with the Sensitization Group, the ear redness-swelling and scabbing of the rMBP-NAP group were significantly ameliorated (see FIG. 3).

2. Histopathological Examination of AD Mice

H&E analysis shows:

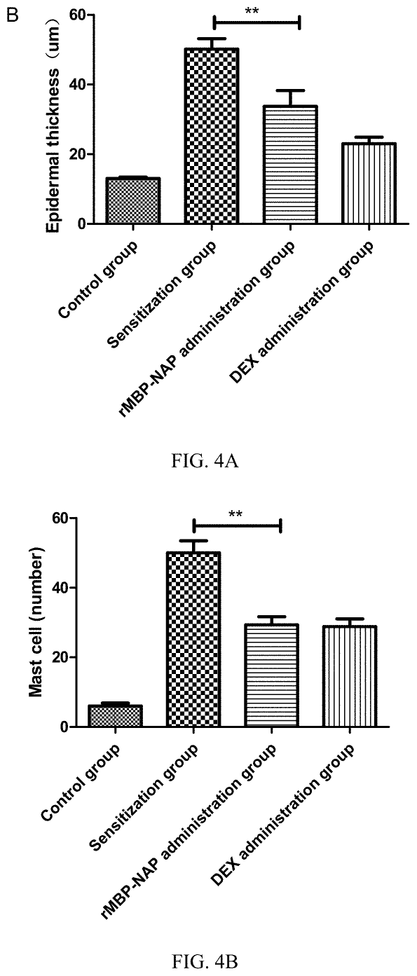

Symptoms occurred in the Sensitization Group comprising that the stratum corneum of the skin was damaged, the dermis was thickened, a large number of inflammatory cells infiltrated, and the blood vessels were dilated. while in the rMBP-NAP Administration Group, the exudation of the inflammatory cells was not obvious, the epidermis was slightly thickened, the stratum corneum was intact, and the blood vessels were not obviously dilated, which suggest that the therapeutic effect of the rMBP-NAP Administration Group is more significant (see FIG. 4A).

The ear tissues were stained with toluidine blue to observe the infiltration of mast cells in the ear tissues of the Sensitization Group. The results showed that in the Sensitization Group, near the ear epidermis layer, there were a large number of purple or purplish red granules(those indicates the mast cells stained). But toluidine blue staining analysis of that in the rMBP-NAP Administration Group showed that the number of the mast cells near the ear epidermis was decreased and the infiltration of the mast cells was reduced. (see FIG. 4B for details).

3. Organ Index Analysis:

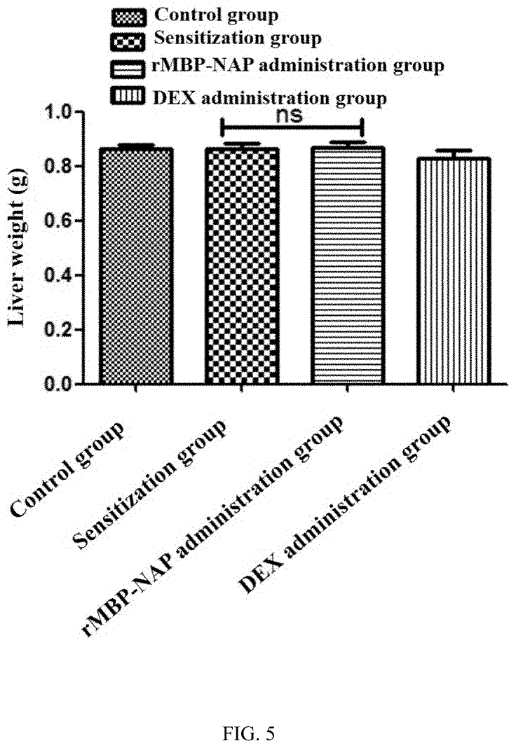

As shown in FIG. 5, the results of liver organ index analysis showed no significant difference between each of the groups.

1. (canceled)

2. A pharmaceutical composition for treating atopic dermatitis, comprising protein(s) and a pharmaceutically acceptable carrier, wherein the protein(s) is one or more proteins selected from the group consisting of HP-NAP and rMBP-NAP.

3. The pharmaceutical composition according to claim 2, wherein the pharmaceutical composition is an injection.

4. The pharmaceutical composition according to claim 3, wherein the pharmaceutical composition is powder for injection or solution for injection.

5. (canceled)

6. A method for treating atopic dermatitis with protein(s), wherein the protein(s) is one or more proteins selected from the group consisting of HP-NAP and rMBP-NAP.

7. The method according to claim 6, wherein the protein(s) is administered by injection.

8. (canceled)

9. (canceled)
